(19) United States
(12) Patent Application Publication
(10) Pub. No.: US 2011/0135590 A1
(43) Pub. Date: Jun. 9, 2011

(54) PRODUCT FOR KERATIN-CONTAINING FIBERS COMPRISING AT LEAST ONE SPECIFIC AMPHIPHILIC, CATIONIC POLYMER, AND AT LEAST ONE POLYOL

(76) Inventors: Thorsten Knappe, Schenefeld (DE); Marcus Noll, Norderstedt (DE)

(21) Appl. No.: 13/027,637

(22) Filed: Feb. 15, 2011

Related U.S. Application Data
(63) Continuation of application No. PCT/EP09/59348, filed on Jul. 21, 2009.

(30) Foreign Application Priority Data
Aug. 18, 2008 (DE) 10 2008 038 112.8

(51) Int. Cl. A61K 8/81; A61K 8/84; A61K 8/88; A61Q 5/06

(52) U.S. Cl. 424/70.15; 424/70.11; 424/70.17

(57) ABSTRACT

Product for treating keratin-containing fibers, especially human hair, containing, in a cosmetically acceptable carrier: (a) at least one amphiphilic, cationic polymer having at least one structural unit according to Formulae (I), (II) (III) and (IV), wherein R¹ and R⁴ are independently a hydrogen atom or a methyl group, X¹ and X² are independently an oxygen atom or an NH group, A¹ and A² are independently an ethane-1,2-diyl, propane-1,3-diyl or butane-1,4-diyl group, R², R³, R⁵ and R⁶ are independently a (C₁-C₄)-alkyl group, R⁷ is a (C₈-C₃₀)-alkyl group, and (b) at least one polyol. The invention also relates to use of the product for temporary hair styling and for hair care, especially in the form of a hair cream or a hair gel.

### CROSS REFERENCE TO RELATED APPLICATIONS

The present application is a continuation of International Patent Application No. PCT/EP2009/059348 filed 21 Jul. 2009, which claims priority to German Patent Application No. 10 2008 038 112.8 filed 18 Aug. 2008, both of which are incorporated herein by reference.

The present invention relates to agents for treating hair comprising a combination of at least one specific amphiphilic, cationic polymer with at least one polyol, use of these agents for temporary shaping and/or for care of keratin-containing fibers, and hair gels based on these agents.

Keratin-containing fibers are understood to include all animal hair (e.g., wool, horsehair, angora hair, furs and feathers) and products or fabrics produced from them. However, keratinic fibers preferably concern human hair.

Today, a suitably looking hairstyle is generally regarded as an essential part of a well groomed appearance. Based on actual fashion trends, hairstyles which are often considered chic can, for many types of hair, only be formed or sustained over a longer period of up to several days by using certain consolidating materials. Thus, hair treatments that provide permanent or temporary hairstyling play an important role. Temporary styling intended to provide a good hold without compromising the healthy appearance of the hair, such as gloss, can be obtained, for example, by use of hairsprays, hair waxes, hair gels, hair foams, setting lotions etc.

Suitable compositions for temporary hairstyling usually contain synthetic polymers as the styling component. Preparations comprising a dissolved or dispersed polymer can be applied on the hair by propellants or by a pumping mechanism. Hair gels and hair waxes are, however, not generally applied directly on the hair, but rather dispersed with a comb or by hand. The most important property of an agent for temporary styling of keratin fibers, herein also referred to as styling agents, consists in giving the treated fibers the strongest possible hold for the shape created. If the keratinic fibers involve human hair, then one also speaks of a strong hairstyle hold or a high degree of hold of the styling agent. Styling hold is basically determined by the type and quantity of synthetic polymer used; however, other components of the styling agent may also influence the hold. In addition to a high degree of hold, styling agents must fulfill a whole series of additional requirements. These requirements can be broadly subdivided into properties on the hair, properties of the formulation in question (e.g., properties of the foam, gel or sprayed aerosol), and properties concerning the handling of the styling agent, wherein particular importance is attached to the properties on the hair. Such properties include moisture resistance, low stickiness and a balanced conditioning effect. Furthermore, a styling agent should be universally applicable for as many types of hair as possible.

To do justice to the various requirements, various synthetic polymers have been developed and are in use in styling agents. These polymers can be subdivided into cationic, anionic, non-ionic and amphoteric film-forming and/or setting polymers. Ideally these polymers form a polymer film when applied to hair, imparting a strong hold to the hairstyle while also being sufficiently flexible so as to not break under stress. If the polymer film is too brittle, film plaques develop (i.e., residues that are shed with movement of the hair and give the impression that the user of the respective styling agent has dandruff). Still, developing styling agents having all the desired properties still presents problems. This particularly applies to the combination of strong and flexible hold. In order to impart a strong hold, the setting polymer has to adhere well to the keratin-containing fibers and form a sufficiently hard film. However, the resulting polymer film should not give the perception of being stiff as a board to the collective fibers, but rather impart a degree of flexibility to the fibers without losing the marked styling of the collective fibers (i.e., the hair style).

Accordingly, the present invention provides an agent for the temporary styling of keratinic fibers, wherein the agent has a very high degree of hold yet still imparts flexibility to the fibers and does not form film plaques.

It has now been surprisingly found that this can be achieved by a combination of a specific polymer with at least one polyol. The combination of active substances brings about an excellent association of a strong hold and a flexible styling.

Accordingly, a first subject matter of the present invention is agents for treating keratin-containing fibers, especially human hair, comprising in a cosmetically acceptable carrier—
(a) at least one amphiphilic, cationic polymer containing at least one structural unit of Formula (I), at least one structural unit of Formula (II), at least one structural unit of Formula (III) and at least one structural unit of Formula (IV),

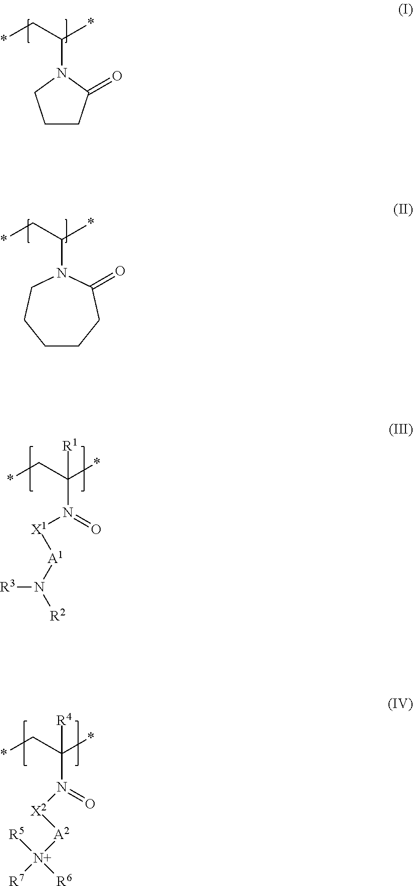

wherein

R¹ and R⁴ are, independently of one another, a hydrogen atom or a methyl group,

X¹ and X² are, independently of one another, an oxygen atom or an NH group,

A¹ and A² are, independently of one another, an ethane-1,2-diyl, propane-1,3-diyl or butane-1,4-diyl group,

R², R³, R⁵ and R⁶ are, independently of one another, a (C₁ to C₄) alkyl group, and

R⁷ is a (C₈ to C₃₀) alkyl group, and
(b) at least one polyol

In the above formulae and all formulae below, the symbol * represents a chemical bond that is a free valence of the corresponding structural fragment.

To compensate for the positive polymer charge, all possible physiologically acceptable anions may be used, such as chloride, bromide, hydrogen sulfate, methyl sulfate, ethyl sulfate, tetrafluoroborate, phosphate, hydrogen phosphate, dihydrogen phosphate or p-toluene sulfonate, triflate.

Exemplary inventive (C₁ to C₄) alkyl groups are methyl, ethyl, propyl, isopropyl, butyl, sec-butyl, isobutyl, tert-butyl.

Exemplary inventive (C₈ to C₃₀) alkyl groups are octyl (capryl), decyl (caprinyl), dodecyl (lauryl), tetradecyl (myristyl), hexadecyl (cetyl), octadecyl (stearyl), eicosyl (arachyl), docosyl (behenyl).

The properties of agents according to the invention have proven to be particularly advantageous when it is in the form of a gel. This preferred packaging form is described later in detail.

The following amphiphilic, cationic polymers are preferably employed in the agents when the amphiphilic, cationic polymers fulfill one or more of the following criteria:
R¹ and R⁴ are each a methyl group,
X¹ is an NH group,
X² is an NH group,
A¹ and A² are each, independently of one another, ethane-1,2-diyl or propane-1,3-diyl,
R², R³, R⁵ and R⁶ are each, independently of one another, methyl or ethyl, (preferably methyl),
R⁷ is a (C₁₀ to C₂₄) alkyl group, especially decyl (caprinyl), dodecyl (lauryl), tetradecyl (myristyl), hexadecyl (cetyl), octadecyl (stearyl), eicosyl (arachyl) or docosyl (behenyl).

Preferably, the structural unit of Formula (III) is chosen from at least one of structural units of Formula (III-1) to (III-8)—

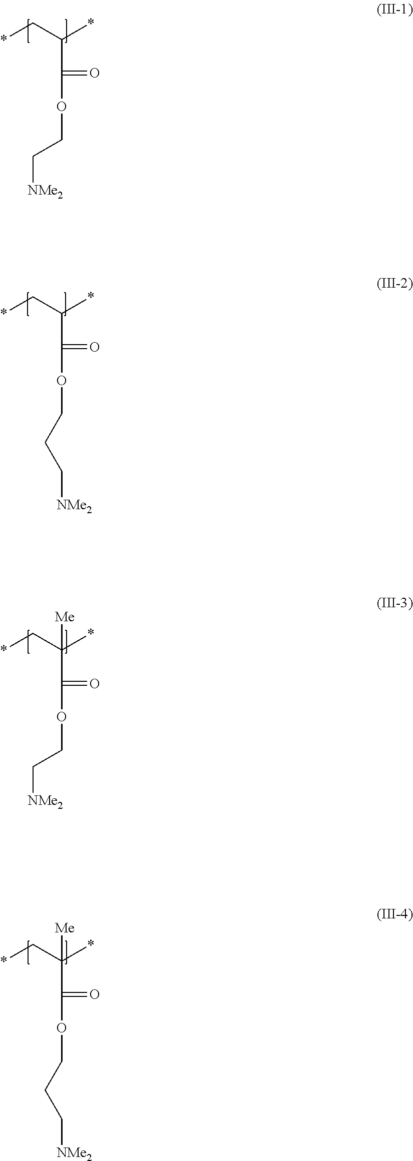

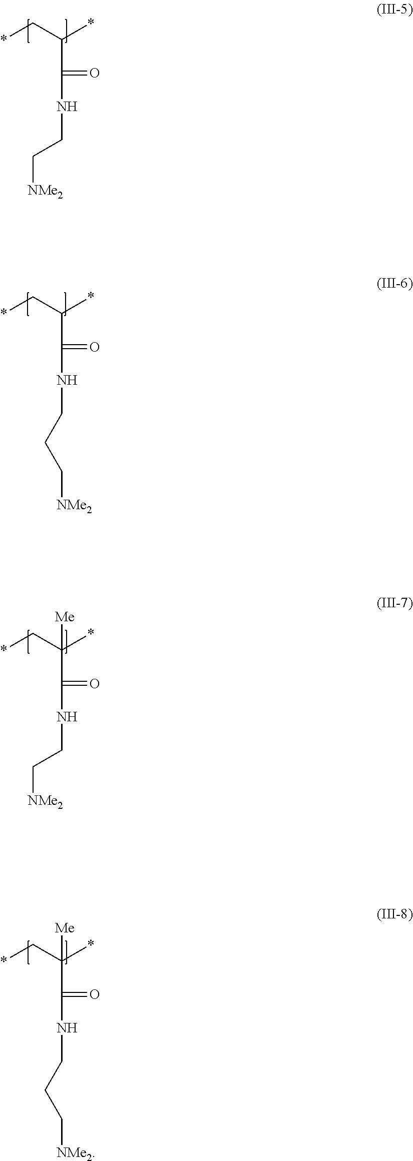

Moreover, it is particularly preferable to choose the structural unit according to Formula (III-7) and/or Formula (III-8) as the structural unit of Formula (III). According to the invention, the structural unit of Formula (III-8) is a quite particularly preferred structural unit. Furthermore, the structural unit of Formula (IV) is preferably chosen from at least one structural unit according to the following Formulae (IV-1) to (IV-8)—

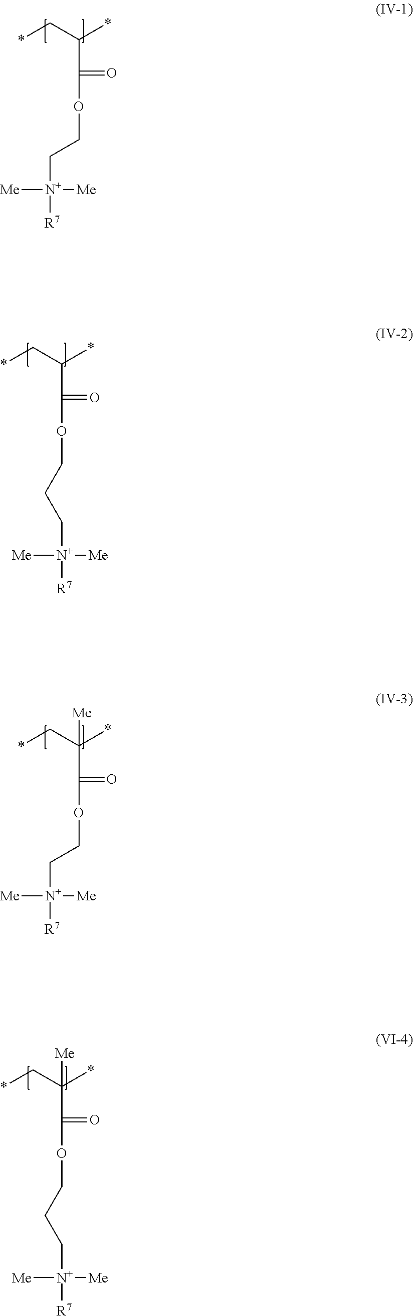

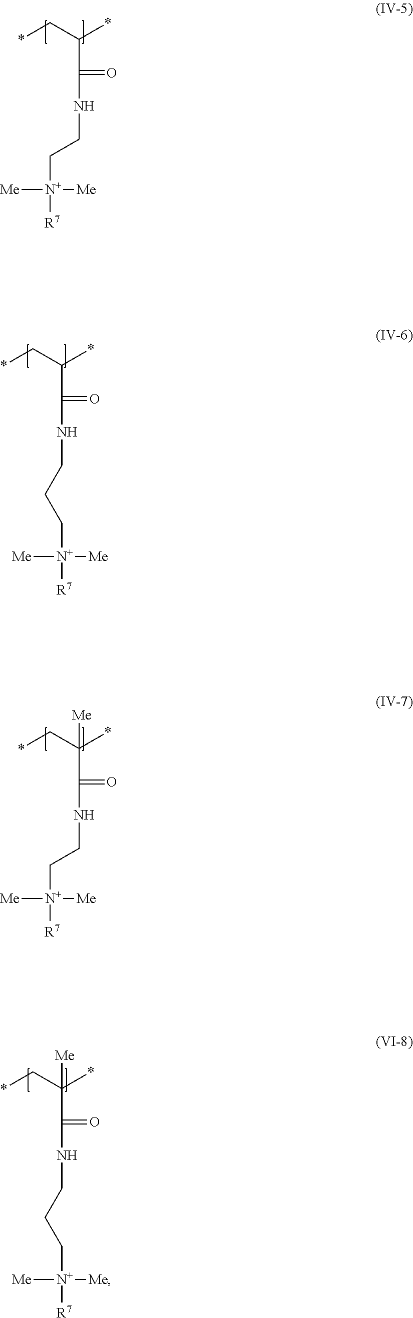

wherein R⁷ is a (C₈ to C₃₀) alkyl group.

Formula (IV-7) and/or Formula (IV-8) structural units are particularly preferred as the structural unit of Formula (IV), wherein R⁷ is octyl (capryl), decyl (caprinyl), dodecyl (lauryl), tetradecyl (myristyl), hexadecyl (cetyl), octadecyl (stearyl), eicosyl (arachyl) or docosyl (behenyl). According to the invention, the structural unit of Formula (IV-8) represents a quite particularly preferred structural unit of Formula (IV).

A quite particularly preferred amphiphilic, cationic polymer contains the following structural units of Formulae (I), (II), (III-8) and (IV-8)—

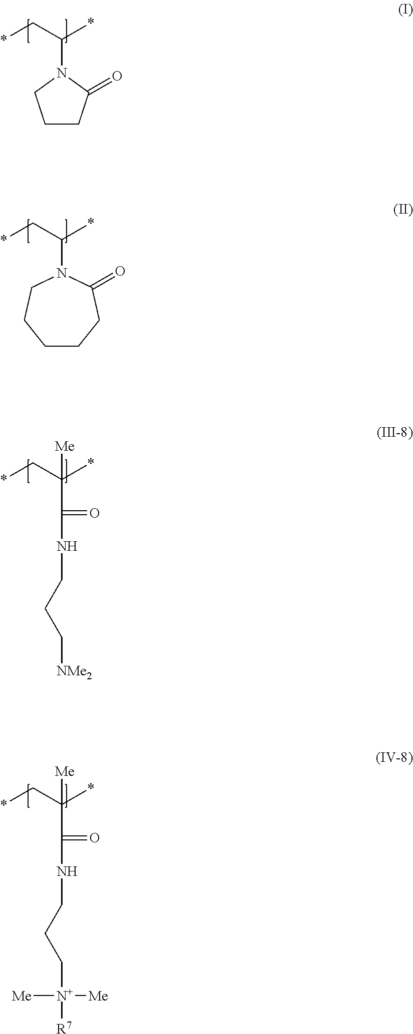

wherein R⁷ is a (C₈ to C₃₀) alkyl group, especially octyl (capryl), decyl (caprinyl), dodecyl (lauryl), tetradecyl (myristyl), hexadecyl (cetyl), octadecyl (stearyl), eicosyl (arachyl) or docosyl (behenyl).

Molecular weights of amphiphilic, cationic polymers according to the invention are preferably from 10,000 g/mol to 50,000,000 g/mol, especially from 50,000 g/mol to 5,000,000 g/mol, particularly preferably from 75,000 g/mol to 1,000,000 g/mol.

A particularly preferred amphiphilic, cationic polymer is the copolymer of N-vinyl pyrrolidone, N-vinyl caprolactam, N-(3-dimethylaminopropyl)methacrylamide and 3-(methacryloylamino)propyl-lauryl-dimethylammonium chloride (INCI name: Polyquatemium-69), marketed, for example, under the trade name AquaStyle® 300 (28-32 wt. % active substance in an ethanol-water mixture) by the ISP company.

In the context of the invention, preferred agents contain the above described amphiphilic, cationic polymers in an amount of 0.05 wt. % to 15.0 wt. %, particularly preferably 0.05 wt. % to 10.0 wt %, quite particularly preferably 0.1 wt. % to 5.0 wt. %, based on total weight of the agent.

Moreover, the agent according to the invention contains, in addition to the previously defined amphiphilic, cationic polymer, at least one polyol.

In the context of the invention, preferred agents contain polyols in an amount of 0.05 wt. % to 15.0 wt. %, particularly preferably 0.05 wt. % to 10.0 wt. %, quite particularly preferably 0.1 wt. % to 5.0 wt. %, based on total weight of the agent.

Polymers (a) and polyols (b) are preferably employed in a weight ratio [polymer (a) to polyol (b)] of 1 to 50 to 50 to 1, particularly 1 to 30 to 30 to 1, more particularly 1 to 10 to 10 to 1.

It is inventively preferred when the polyol is solid at 25° C. under a pressure of 1 atm.

Furthermore, preferred polyols have a chemical structure that is made exclusively of carbon atoms, hydrogen atoms and oxygen atoms, and have a hydrocarbon backbone onto which at least three hydroxyl groups (especially four hydroxyl groups) are bonded. In this respect, the oxygen atoms preferably bond solely as hydroxyl groups (—OH) and/or as ether functionalities (—O—) onto the hydrocarbon backbone.

According to an inventively preferred embodiment, molecules of polyols (b) have at least 0.8 hydroxyl groups, preferably at least 0.9 hydroxyl groups, more preferably 1 hydroxyl group per carbon atom of the hydrocarbon backbone.

Furthermore, it is inventively preferred when all oxygen atoms in the molecule of the linear aliphatic polyol are in the form of hydroxyl groups.

Moreover, in the context of the invention, those polyols (b) are preferably suitable whose molar carbon to oxygen ratio is in the range 0.8 to 1.5, particularly 0.9 to 1.2, quite preferably 1.

However, it is also preferable when the molar ratio of carbon atoms to oxygen atoms in the molecule of the polyol (b) is 1, and the molar ratio of hydrogen atoms to oxygen atoms in the molecule of the polyol (b) is greater than 2.

Preferably, polyol (b) is chosen from sorbitol, inositol, xylitol, mannitol, gluconolactone, glucuronic acid, 1,2,6-hexane triol, hydroxyethylsorbitol, phytantriol, hydroxypropylphytantriol, lactitol, maltitol, pentaerythritol, polyglycerin-3, glucose, lactose, maltose, polyglycerin-4, polyglycerin-6, polyglycerin-10, polyglycerin-20, polyglycerin-40, tripropylene glycol. It is particularly preferred when polyol (b) is chosen from sorbitol, inositol, xylitol, mannitol, hydroxyethylsorbitol, phytantriol, hydroxypropylphytantriol, lactitol, maltitol, pentaerythritol, polyglycerin-3, polyglycerin-4, polyglycerin-6, polyglycerin-10, polyglycerin-20, polyglycerin-40. A quite particularly preferred polyol (b) is sorbitol.

A quite particularly preferred agent has, in a cosmetically acceptable carrier—
(a) at least one amphiphilic, cationic polymer containing structural units according to the following Formulae (I), (II), (III-8) and (IV-8)

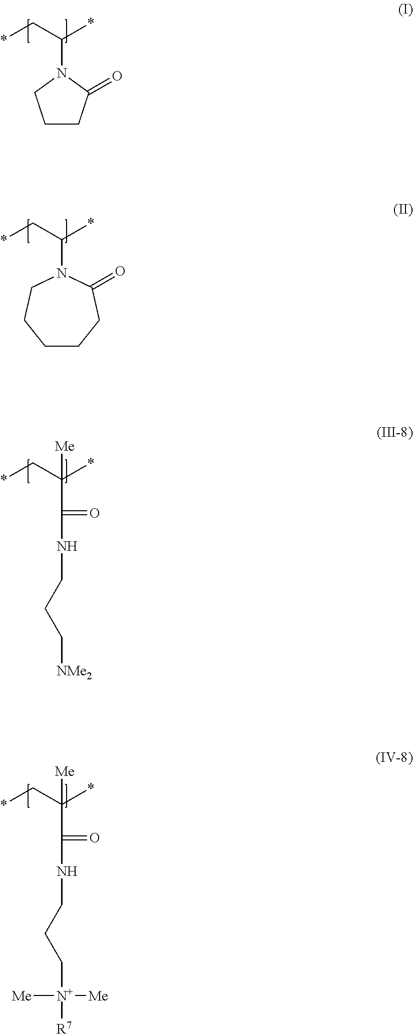

wherein R⁷ is a (C₈ to C₃₀) alkyl group (especially octyl (capryl), decyl (caprinyl), dodecyl (lauryl), tetradecyl (myristyl), hexadecyl (cetyl), octadecyl (stearyl), eicosyl (arachyl) or docosyl (behenyl)), and
(b) sorbitol.

In a preferred embodiment, the agent further comprises, in addition to the amphiphilic, cationic polymer (a) and polyol (b), at least one film-forming and/or setting polymer (c). The latter polymer (c) differs from polymer (a).

Preferred properties of the film-forming polymers (c) include film formation. Film-forming polymers refer to those polymers that, on drying, leave a continuous film on the skin, hair or nails. These types of film-former can be used in a wide variety of cosmetic products such as make up masks, make up, hair sets, hair sprays, hair gels, hair waxes, hair conditioners, shampoos or nail varnishes. Particularly preferred polymers are sufficiently soluble in alcohol or water/alcohol mixtures, so that they are present in completely dissolved form in the agents. The film-forming polymers can be of synthetic or of natural origin.

According to the invention, film-forming polymers further refer to those polymers that, when used in concentrations of 0.01 to 20 wt. % in aqueous, alcoholic or aqueous alcoholic solution, are able to separate out a transparent polymer film on the hair.

Setting polymers contribute to hold and/or creation of hair volume and hair body of the whole hairstyle. These polymers are also film-forming polymers and therefore are typical substances for styling hair treatment compositions, such as hair sets, hair foams, hair waxes, and hair sprays. Film formation can be in completely selected areas and bond only some fibers together.

The curl-retention test is frequently used as a test method for the setting action.

As polymers are often multifunctional (i.e., show a plurality of desired end-use effects), a large number of polymers are found in many of the groups subdivided according to the mode of action, therefore also in the CTFA Handbook.

The agent preferably has at least one film-forming and/or setting polymer chosen from at least one polymer of the group of non-ionic polymers, cationic polymers, amphoteric polymers, zwitterionic polymers and anionic polymers.

Agents according to the invention preferably contain additional film-forming and/or setting polymers in an amount of 0.01 wt. % to 20.0 wt. %, particularly preferably 0.5 wt. % to 15.0 wt. %, quite particularly preferably 2.0 wt. % to 10.0 wt. %, based on total weight of the agent. These amounts also apply for all subsequent preferred types of film-forming and/or setting polymers that can be used in the inventive agents. When subsequently different preferred quantities are specified, then the latter are to be again taken as the preferred quantities.

Particularly suitable agents contain, besides the previously defined amphiphilic, cationic polymers, at least one film-forming and/or setting polymer chosen from at least one polymer of the group consisting of

non-ionic polymers based on ethylenically unsaturated monomers, especially
homopolymers of N-vinyl pyrrolidone,
non-ionic copolymers of N-vinyl pyrrolidone,
homopolymers and non-ionic copolymers of N-vinyl caprolactam,
copolymers of (meth)acrylamide, and
polyvinyl alcohol, polyvinyl acetate,

chitosan and derivatives of chitosan,

cationic cellulose derivatives,

cationic copolymers of 3-(C₁ to C₆) alkyl-1-vinyl-imidazolinium,

homopolymers and copolymers comprising the structural unit of Formula (M-1)

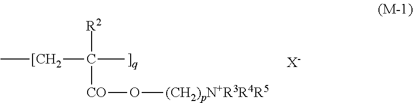

wherein R²=—H or —CH₃; R³, R⁴ and R⁵ are, independently of each other, (C₁ to C₄) alkyl, (C₁ to C₄) alkenyl, or (C₂ to C₄) hydroxyalkyl groups; p=1, 2, 3 or 4; q is a natural number; and X⁻ is a physiologically acceptable organic or inorganic anion;

anionic polymers that exhibit carboxylate and/or sulfonate groups; and/or

anionic polyurethanes.

Preferred non-ionic polymers based on ethylenically unsaturated monomers that are suitable as additional film-forming and/or setting polymers are those containing at least one of the following structural units—

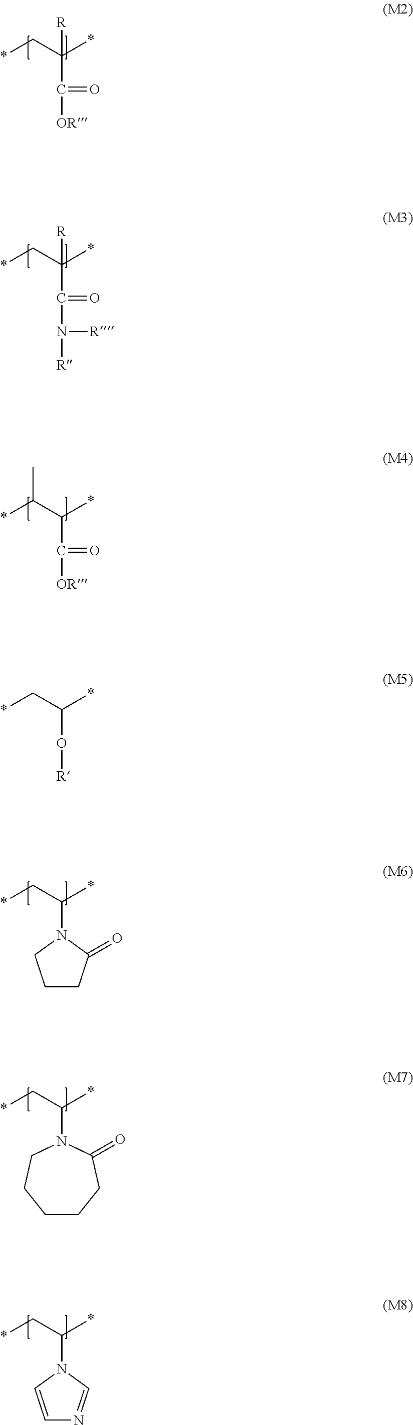

wherein
R is a hydrogen atom or a methyl group,
R′ is a hydrogen atom or a (C₁ to C₄) acyl group,
R″ and R″″ are, independently of one another, a (C₁ to C₇) alkyl group or a hydrogen atom, and
R″″ is a linear or branched (C₁ to C₄) alkyl group or a (C₂ to C₄) hydroxyalkyl group.

Suitable, non-ionic film-forming and/or non-ionic hair setting polymers include homopolymers or copolymers based on at least one of the following monomers: vinyl pyrrolidone, vinyl caprolactam, vinyl esters such as vinyl acetate, vinyl alcohol, acrylamide, methacrylamide, alkyl and dialkyl acrylamide, alkyl and dialkyl methacrylamide, alkyl acrylate, alkyl methacrylate, wherein the alkyl groups of these monomers are chosen from (C₁ to C₃) alkyl groups.

Particularly suitable non-ionic polymers based on ethylenically unsaturated monomers for agents according to the invention have at least one of the following structural units—

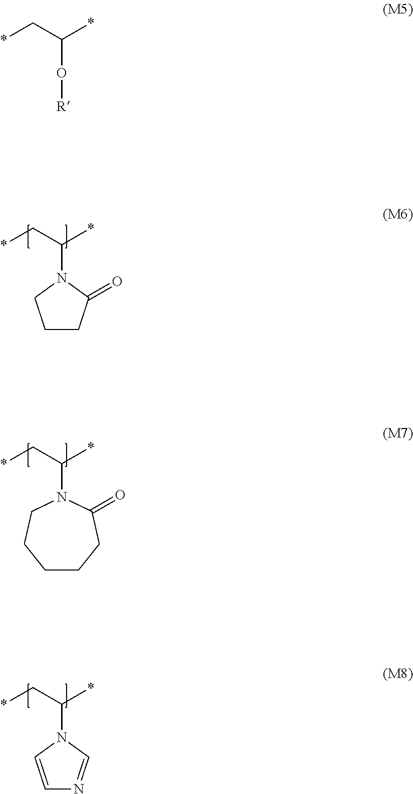

wherein R′ is a hydrogen atom or a (C₁ to C₃₀) acyl group, particularly a hydrogen atom or an acetyl group.

Homopolymers of vinyl caprolactam or vinyl pyrrolidone (such as Luviskol® K 90 or Luviskol® K 85 from BASF SE), copolymers of vinyl pyrrolidone and vinyl acetate (such as those marketed under the trade names Luviskol® VA 37, Luviskol® VA 55, Luviskol® VA 64 and Luviskol® VA 73 by BASF SE), terpolymers of vinyl pyrrolidone, vinyl acetate and vinyl propionate, polyacrylamides (such as Akypomine® P 191 from CHEM-Y), polyvinyl alcohols (marketed, for example, under the trade names Elvanol® by Du Pont or Vinol® 523/540 by Air Products), terpolymers of vinyl pyrrolidone, methacrylamide and vinyl imidazole (such as Luviset® Clear from BASF SE) are particularly suitable.

In addition to non-ionic polymers based on ethylenically unsaturated monomers, non-ionic cellulose derivatives are also suitable film-forming and/or setting polymers for the preferred achievement of the technical teaching. They are preferably chosen from methyl cellulose, and especially from cellulose ethers such as hydroxypropyl cellulose (e.g., hydroxypropyl cellulose with a molecular weight of 30,000 to 50,000 g/mol, marketed, for example, under the trade name Nisso SI® by Lehmann & Voss, Hamburg), hydroxyethyl celluloses, such as those marketed under the trade names Culminal® and Benecel® (AQUALON) and Natrosol® types (Hercules).

Particularly preferred agents according to the invention contain as an additional film-forming and/or setting polymer a non-ionic polymer chosen from homopolymers of N-vinyl pyrrolidone and/or copolymers of N-vinyl pyrrolidone. Agents having as the film-forming non-ionic and/or setting non-ionic polymer at least one polymer chosen from—
polyvinyl pyrrolidone (most preferred),
copolymers of N-vinyl pyrrolidone and vinyl esters of carboxylic acids containing 2 to 8 carbon atoms, especially from N-vinyl pyrrolidone and vinyl acetate,
copolymers of N-vinyl pyrrolidone and N-vinyl imidazole and methacrylamide,
copolymers of N-vinyl pyrrolidone and N-vinyl imidazole and acrylamide, and
copolymers of N-vinyl pyrrolidone with N,N-di(C₁ to C₄) alkylamino (C₂ to C₄) alkylacrylamide,
   are inventively quite particularly preferred.

A particularly preferred agent comprises, in a cosmetically acceptable carrier—
(a) at least one amphiphilic, cationic polymer containing structural units according to the following Formulae (I), (II), (III-8) and (IV-8)

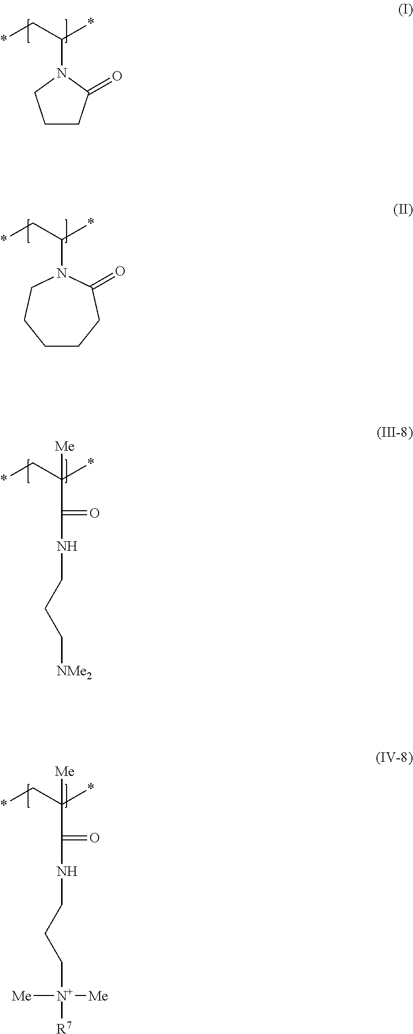

wherein R⁷ is a (C₈ to C₃₀) alkyl group (especially octyl (capryl), decyl (caprinyl), dodecyl (lauryl), tetradecyl (myristyl), hexadecyl (cetyl), octadecyl (stearyl), eicosyl (arachyl) or docosyl (behenyl)),
(b) at least one polyol, and
(c) polyvinyl pyrrolidone.

A quite particularly preferred agent comprises, in a cosmetically acceptable carrier—
(a) at least one amphiphilic, cationic polymer containing the following structural units according to Formulae (I), (II), (III-8) and (IV-8)

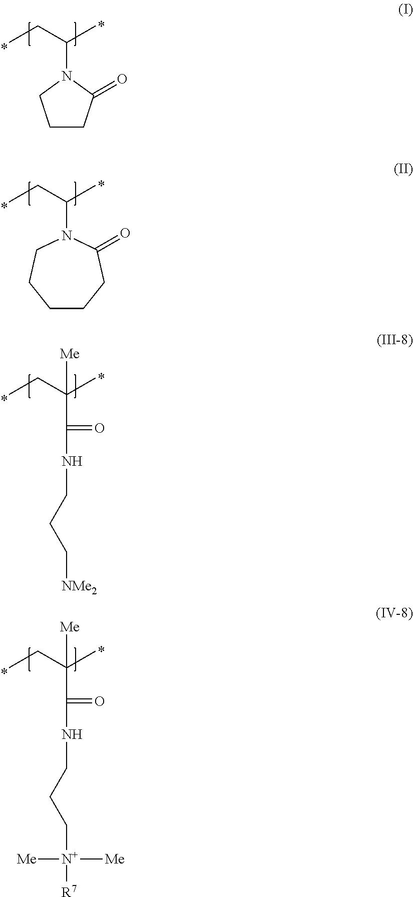

wherein R⁷ is a (C₈ to C₃₀) alkyl group (especially octyl (capryl), decyl (caprinyl), dodecyl (lauryl), tetradecyl (myristyl), hexadecyl (cetyl), octadecyl (stearyl), eicosyl (arachyl) or docosyl (behenyl)),
(b) sorbitol, and
(c) polyvinyl pyrrolidone.

Cationic polymers refer to polymers that, in their main chain and/or side chain, possess groups that can be “temporarily” or “permanently” cationic. “Permanently cationic” refers, according to the invention, to those polymers that, independently of the pH of the medium, have a cationic group. These are, for example, polymers typically having a quaternary nitrogen atom in the form of an ammonium group. Preferred cationic groups are quaternary ammonium groups. In particular, those polymers in which the quaternary ammonium groups are bonded through a C1-4 hydrocarbon group to a polymer backbone formed from acrylic acid, methacrylic acid or their derivatives have proved to be particularly suitable.

An inventively preferred suitable cationic film-forming and/or cationic setting polymer is at least one cationic film-forming and/or cationic setting polymer having at least one structural element of Formula (M9) and additionally at least one structural element of Formula (M10)

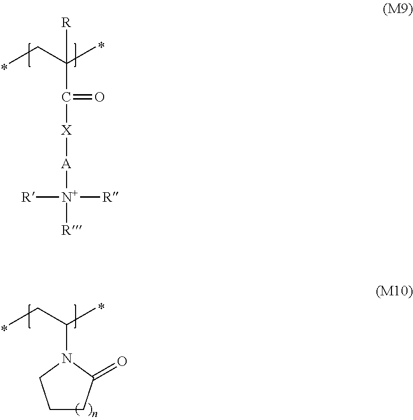

wherein
R is a hydrogen atom or a methyl group,
R′, R″ and R″ are, independently of one another, a (C₁ to C₃₀) alkyl group,
X is an oxygen atom or an NH group,
A is an ethane-1,2-diyl group or a propane-1,3-diyl group, and
n is 1 or 3.

To compensate for the positive polymer charge, all possible physiologically acceptable anions may be used, such as chloride, bromide, hydrogen sulfate, methyl sulfate, ethyl sulfate, tetrafluoroborate, phosphate, hydrogen phosphate, dihydrogen phosphate or p-toluene sulfonate, triflate.

Such compounds include copolymers of dimethylaminoethyl methacrylate, quaternized with diethyl sulfate, with vinyl pyrrolidone having the INCI name Polyquaternium-11 and commercially available under the trade names Gafquat® 440, Gafquat® 734, Gafquat® 755 (each from ISP) and Luviquat PQ 11 PN (BASF SE).

Furthermore, cationic film-forming and/or cationic setting polymers are particularly preferably chosen from cationic, quaternized cellulose derivatives.

Moreover, cationic, quaternized cellulose derivatives are preferred suitable film-forming and/or setting polymers.

Those cationic, quaternized celluloses having more than one permanent cationic charge in a side chain have proven to be particularly advantageous in the context of the invention. Among these cationic celluloses, those having the INCI name Polyquatemium-4 are particularly suitable, which, for example, are marketed by National Starch and Chemical Company under the trade names Celquat® H 100, Celquat® L 200.

In the context of the invention, those cationic film-forming and/or cationic setting copolymers having at least one structural element according to Formula (M11) additionally serve as the particularly preferred usable cationic polymers

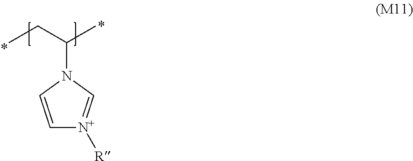

wherein R″ is a (C₁ to C₄) alkyl group, particularly a methyl group,
and additionally possess at least one further cationic and/or non-ionic structural element.

To compensate for the positive polymer charge, all possible physiologically acceptable anions may be used, such as chloride, bromide, hydrogen sulfate, methyl sulfate, ethyl sulfate, tetrafluoroborate, phosphate, hydrogen phosphate, dihydrogen phosphate or p-toluene sulfonate, triflate.

It is again preferred when at least one copolymer (c1) containing, in addition to at least one structural element of Formula (M11), a structural element of Formula (M6), is comprised as the additional cationic film-forming and/or cationic setting polymer

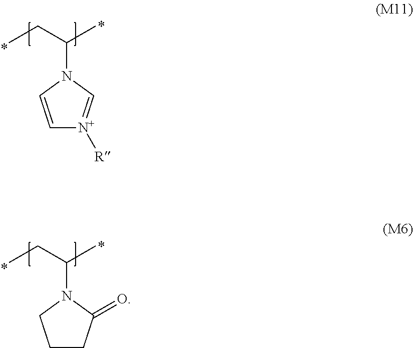

wherein R″ is a (C₁ to C₄) alkyl group, particularly a methyl group.

To compensate for the positive polymer charge of the copolymer (c1), all possible physiologically acceptable anions may be used, such as chloride, bromide, hydrogen sulfate, methyl sulfate, ethyl sulfate, tetrafluoroborate, phosphate, hydrogen phosphate, dihydrogen phosphate or p-toluene sulfonate, triflate.

Cationic film-forming and/or cationic setting polymers that are quite particularly preferred as copolymers (c1) contain 10 to 30 mol %, preferably 15 to 25 mol % and particularly 20 mol % of structural units in accordance with Formula (M11) and 70 to 90 mol %, preferably 75 to 85 mol % and particularly 80 mol % of structural units in accordance with Formula (M6).

In this regard it is particularly preferred when copolymers (c1) contain, in addition to polymer units resulting from incorporation of the cited structural units according to Formulae (M11) and (M6) into the copolymer, a maximum of 5 wt. %, preferably a maximum of 1 wt. % of polymer units that trace back to the incorporation of other monomers. Copolymers (c1) are preferably exclusively formed from structural units of Formula (M11) with R″=methyl and (M6) and can be described by the general Formula (Poly1)

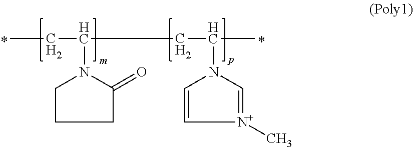

wherein each of the indices m, and p vary according to the molecular mass of the polymer and are not intended to portray block copolymers. In fact, structural units of Formula (M11) and Formula (M6) can be statistically distributed in the molecule.

If a chloride ion is used to compensate the positive charge of the polymer of Formula (Poly1), then these N-methyl vinyl imidazole/vinyl pyrrolidone copolymers are named according to INCI nomenclature as Polyquaternium-16 and are available from, for example, BASF under the trade names Luviquat® Style, Luviquat® FC 370, Luviquat® FC 550, Luviquat® FC 905 and Luviquat® HM 552. If a methosulfate ion is used to compensate the positive charge of the polymer of Formula (Poly1), then these N-methyl vinylimidazole/vinyl pyrrolidone copolymers are named according to INCI nomenclature as Polyquaternium-44 and are available from, for example, BASF under the trade name Luviquat® UltraCare.

Particularly preferred inventive agents contain a copolymer (c1), especially of Formula (Poly1), which has molecular masses within a defined range. Here, inventive agents are preferred wherein the molecular mass of copolymer (c1) is from 50 to 400 kDa, preferably from 100 to 300 kDa, more preferably from 150 to 250 kDa and particularly from 190 to 210 kDa.

In addition to copolymer(s) (c1) or instead of it or them, the agents can also contain copolymers (c2) that, starting from the copolymer (c1), possess as the additional structural units structural units of Formula (M7)

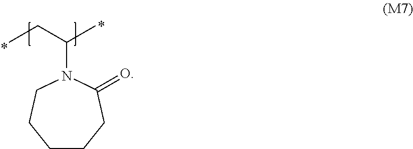

Further particularly preferred agents according to the invention are accordingly those having as the cationic film-forming and/or cationic setting polymer at least one copolymer (c2) containing at least one structural unit according to Formula (M11-a) and at least one structural unit according to Formula (M6) and at least one structural unit according to Formula (M7)

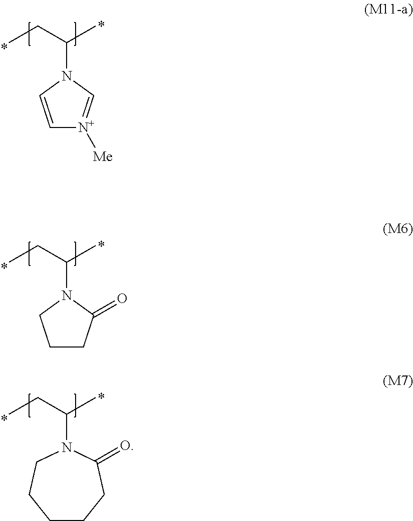

Also, in this regard it is particularly preferred when copolymers (c2) contain, in addition to polymer units resulting from the incorporation of the cited structural units according to Formulae (M11-a), (M6) and (M7) into the copolymer, a maximum of 5 wt. %, preferably a maximum of 1 wt. % of polymer units that trace back to the incorporation of other monomers. The copolymers (c2) are preferably exclusively formed from structural units of Formula (M11-a), (M6) and (M7) and can be described by the general Formula (Poly2)

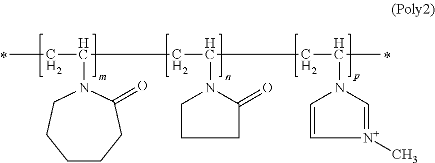

wherein each of indices m, n and p vary according to the molecular mass of the polymer and are not intended to portray block copolymers. In fact, structural units of the cited Formulae can be statistically distributed in the molecule.

To compensate for the positive polymer charge of component (c2), all possible physiologically acceptable anions may be used, such as chloride, bromide, hydrogen sulfate, methyl sulfate, ethyl sulfate, tetrafluoroborate, phosphate, hydrogen phosphate, dihydrogen phosphate or p-toluene sulfonate, triflate.

If a methosulfate ion is used to compensate the positive charge of the polymer of Formula (Poly2), then these N-methyl vinylimidazole/vinyl pyrrolidone/vinyl caprolactam copolymers are named according to INCI nomenclature as Polyquaternium-46 and are available, for example, from BASF under the trade name Luviquat® Hold.

Quite particularly preferred copolymers (c2) contain 1 to 20 mol %, preferably 5 to 15 mol % and particularly 10 mol % of structural units according to Formula (M11-a) and 30 to 50 mol %, preferably 35 to 45 mol % and particularly 40 mol % of structural units according to Formula (M6) and 40 to 60 mol %, preferably 45 to 55 mol % and particularly 60 mol % of structural units according to Formula (M7).

Particularly preferred inventive agents contain a copolymer (c2) having molecular masses within a defined range. Here, inventive agents are preferred wherein the molecular mass of the copolymer (c2) is from 100 to 1000 kDa, preferably from 250 to 900 kDa, more preferably from 500 to 850 kDa and particularly from 650 to 710 kDa.

In addition to copolymer(s) (c1) and/or (c2) or in its or their place, the agents can also contain as the film-forming cationic and/or setting cationic polymer copolymers (c3) having as the structural units structural units of Formulae (M11-a) and (M6), as well as structural units from the group of vinyl imidazole units and further structural units from the group of acrylamide and/or methacrylamide units.

Further particularly preferred agents according to the invention contain as the additional cationic film-forming and/or cationic setting polymer at least one copolymer (c3) having at least one structural unit according to Formula (M11-a) and at least one structural unit according to Formula (M6) and at least one structural unit according to Formula (M10) and at least one structural unit according to Formula (M12)

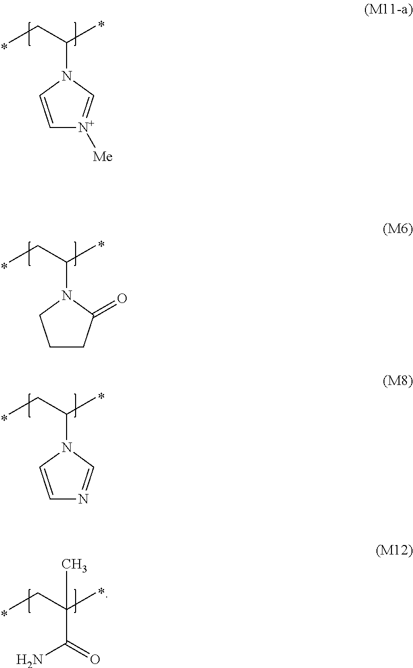

Also, it is particularly preferred when the copolymers (c3) contain, in addition to polymer units resulting from the incorporation of the cited structural units according to Formulae (M11-a), (M6), (M8) and (M12) into the copolymer, a maximum of 5 wt. %, preferably a maximum of 1 wt. % of polymer units that trace back to the incorporation of other monomers. Copolymers (c3) are preferably exclusively formed from structural units of Formula (M11-a), (M6), (M8) and (M12) and can be described by the general Formula (Poly3)

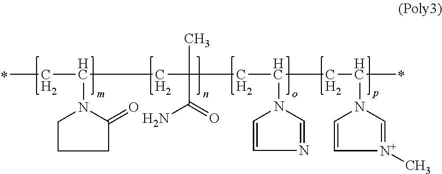

wherein each of indices m, n, o and p vary according to molecular mass of the polymer and are not intended to portray block copolymers. In fact, structural units of Formula (M11-a), (M6), (M8) and (M12) can be statistically distributed in the molecule.

To compensate for the positive polymer charge of the component (c3), all possible physiologically acceptable anions may be used, such as chloride, bromide, hydrogen sulfate, methyl sulfate, ethyl sulfate, tetrafluoroborate, phosphate, hydrogen phosphate, dihydrogen phosphate or p-toluene sulfonate, triflate.

If a methosulfate ion is used to compensate the positive charge of the polymer of Formula (Poly3), then these N-methyl vinyl imidazole/vinyl pyrrolidone/vinyl imidazole/methacrylamide copolymers are named according to INCI nomenclature as Polyquaternium-68 and are available, for example, from BASF under the trade name Luviquat® Supreme.

Quite particularly preferred copolymers (c3) contain 1 to 12 mol %, preferably 3 to 9 mol % and particularly 6 mol % of structural units according to Formula (M11-a) and 45 to 65 mol %, preferably 50 to 60 mol % and particularly 55 mol % of structural units according to Formula (M6) and 1 to 20 mol %, preferably 5 to 15 mol % and particularly 10 mol % of structural units according to Formula (M8) and 20 to 40 mol %, preferably 25 to 35 mol % and particularly 29 mol % of structural units according to Formula (M12).

Particularly preferred inventive agents contain a copolymer (c3) having molecular masses within a defined range. Here, preferred agents are those wherein the molecular mass of the copolymer (c3) is from 100 to 500 kDa, preferably from 150 to 400 kDa, more preferably from 250 to 350 kDa and particularly from 290 to 310 kDa.

Preferred additional film-forming cationic and/or setting polymers, chosen from cationic polymers having at least one structural element of the above Formula (M11-a), include: vinyl pyrrolidone/1-vinyl-3-methyl-1H-imidazolium chloride copolymers (such as that with the INCI name Polyquaternium-16 sold under the trade names Luviquat® Style, Luviquat® FC 370, Luviquat® FC 550, Luviquat® FC 905 and Luviquat® HM 552 (BASF SE)), vinyl pyrrolidone/1-vinyl-3-methyl-1H-imidazolium methylsulfate copolymers (such as that with the INCI name Polyquaternium-44 sold under the trade name Luviquat® Care (BASF SE)), vinyl pyrrolidone/vinyl caprolactam/1-vinyl-3-methyl-1H-imidazolium terpolymer (such as that with the INCI name Polyquatemium-46 sold under the trade names Luviquat® Care or Luviquat® Hold (BASF SE)), vinyl pyrrolidone/methacrylamide/vinyl imidazole/1-vinyl-3-methyl-1H-imidazolium methyl sulfate copolymer (such as that with the INCI name Polyquatemium-68 sold under the trade name Luviquat® Supreme (BASF SE)), as well as mixtures of these polymers.

Further preferred cationic polymers that can be used in the inventive agents are the “temporarily cationic” polymers. These polymers usually contain an amino group that is present at specific pH values as a quaternary ammonium group and is thus cationic. These polymers include chitosan. In the present invention, chitosan and/or chitosan derivatives are considered as quite particularly preferred suitable film-forming and/or setting polymers. Chitosans are biopolymers and are considered to be in the group of the hydrocolloids. From a chemical point of view, they are partially deacetylated chitins of different molecular weight. Those types preferably employed have an average molecular weight of 800,000 to 1,200,000 Dalton, a Brookfield viscosity (1 wt. % conc. in glycolic acid) below 5000 mPas, a deacetylation degree in the range 80 to 88% and an ash content of less than 0.3 wt. %. In the scope of the invention, besides the chitosans as typical cationic biopolymers, cationically derivatized chitosans can also be considered (such as quaternized products) or alkoxylated chitosans.

Inventively preferred agents contain neutralization products of chitosan neutralized with at least one acid and chosen from lactic acid, pyrrolidone carboxylic acid, nicotinic acid, hydroxy-iso-butyric acid, hydroxy-iso-valeric acid, or contain mixtures of these neutralization products as the chitosan derivative(s). Exemplary suitable chitosan (derivatives) are freely available on the market under the trade names Hydagen® CMF (1 wt. % active substance in aqueous solution with 0.4 wt. % glycolic acid, molecular weight 500,000 to 5,000,000 g/mol Cognis), Hydagen® HCMF (chitosan (80% deacetylated), molecular weight 50,000 to 1,000,000 g/mol, Cognis), Kytamer® PC (80 wt. % active substance of chitosan pyrrolidone carboxylate (INCI name: Chitosan PCA), Amerchol) and Chitolam® NB/101.

Agents according to the invention preferably contain chitosan or its derivatives in an amount of 0.01 wt. % to 20.0 wt. %, particularly preferably 0.01 wt. % to 10.0 wt. %, quite particularly preferably 0.1 wt. % to 1 wt. %, based on total weight of the agent.

In the context of the invention, preferred suitable temporarily cationic polymers include those having at least one structural unit according to Formulae (M1-1) to (M1-8)

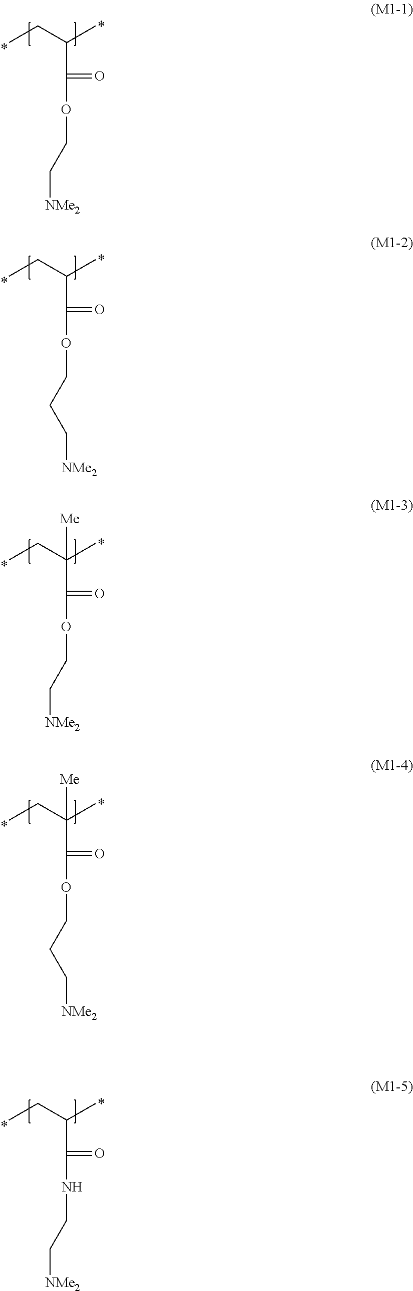

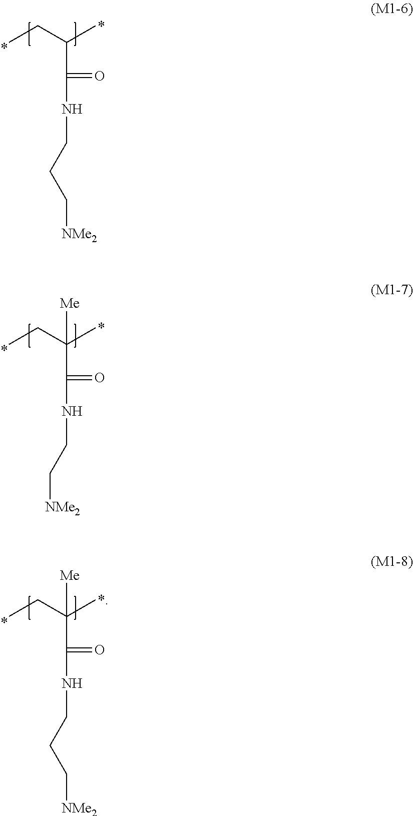

Here, preferred copolymers again have at least one structural unit of Formulae (M1-1) to (M1-8) and additionally at least one structural unit of Formula (M10),

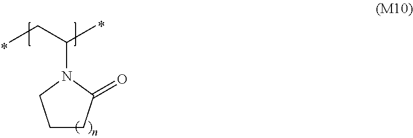

wherein n is 1 or 3.

Here again, the group of polymers
vinyl caprolactam/vinyl pyrrolidone/dimethylaminoethyl methacrylate copolymer (for example INCI name: Vinyl Caprolactam/PVP/Di-methylaminoethyl Methacrylate Copolymer under the trade name Gaffix® VC 713 (ISP)),
vinyl pyrrolidone/vinyl caprolactam/dimethylaminopropylmethacrylamide copolymer (for example INCI name: VP/Vinyl Caprolactam/DMAPA Acrylates Copolymer under the trade name Aquaflex® SF-40 (ISP)),
vinyl caprolactam/vinyl pyrrolidone/dimethylaminoethyl methacrylate copolymer (for example as a 35-39% solids in ethanol in the form of the commercial product Advantage LC E with the INCI name: Vinyl Caprolactam/VP/Dimethylaminoethyl Methacrylate Copolymer, Alcohol, Lauryl Pyrrolidone (ISP)),
vinyl pyrrolidone/dimethylaminopropylmethacrylamide copolymer (for example INCI name: VP/DMAPA Acrylates Copolymer under the trade name Styleze® CC-10 (ISP)),
   forms the preferred list for selection.

Agents according to the invention can also include at least one amphoteric polymer as the film-forming and/or setting polymer. The term amphopolymers includes not only those polymers whose molecule has both free amino groups and free —COOH or SO₃H groups and which are capable of forming inner salts, but also zwitterionic polymers whose molecule has quaternary ammonium groups and —COO or —SO₃ groups, and polymers comprising —COOH or SO₃H groups and quaternary ammonium groups.

An example of a useful amphopolymer according to the invention is the acrylic resin obtainable under the designation Amphomer®, which is a copolymer of tert-butylaminoethyl methacrylate, N-(1,1,3,3-tetramethylbutyl)acrylamide, and two or more monomers from the group consisting of acrylic acid, methacrylic acid and their simple esters.

The latter, in addition to the cationogenic group or the positively charged group, have at least one negatively charged group in the molecule and are also called zwitterionic polymers. Agents according to the invention preferably contain amphoteric polymers in amounts of 0.01 to 20 wt. %, particularly preferably 0.05 to 10 wt. %, based on total weight of the agent. Quantities of 0.1 to 5% by weight are quite particularly preferred.

Furthermore, at least one anionic film-forming and/or anionic setting polymer can be used as the film-forming and/or setting polymers.

Anionic polymers refer to anionic polymers having carboxylate and/or sulfonate groups. Exemplary anionic monomers from which such polymers can be made are acrylic acid, methacrylic acid, crotonic acid, maleic anhydride and 2-acrylamido-2-methylpropane sulfonic acid. Here, the acidic groups may be fully or partially present as sodium, potassium, ammonium, mono- or triethanolammonium salts.

Within this embodiment, it can be preferred to use copolymers of at least one anionic monomer and at least one non-ionic monomer. Regarding the anionic monomers, reference is made to the abovementioned substances. Preferred non-ionic monomers are acrylamide, methacrylamide, acrylic acid esters, methacrylic acid esters, vinyl pyrrolidone, vinyl ethers and vinyl esters.

Preferred anionic copolymers are acrylic acid-acrylamide copolymers, particularly polyacrylamide copolymers with monomers containing sulfonic acid groups. A particularly preferred anionic copolymer contains 70 to 55 mole % acrylamide and 30 to 45 mole % 2-acrylamido-2-methylpropane sulfonic acid, wherein the sulfonic acid group may be fully or partially present as the sodium, potassium, ammonium, mono- or triethanolammonium salt. This copolymer can also be crosslinked, wherein preferred crosslinking agents include polyolefinically unsaturated compounds such as tetraallyloxyethane, allyl sucrose, allyl pentaerythritol and methylene bisacrylamide. Such a polymer is commercially available as the product Sepigel®305 from the SEPPIC Company. Use of this compound, which contains a mixture of hydrocarbons (C₁₃-C₁₄ isoparaffins) and a non-ionic emulsifier (Laureth-7) in addition to the polymer components, has proved to be particularly advantageous in the context of the inventive teaching.

Sodium acryloyl dimethyl taurate copolymers commercially available as a compound with isohexadecane and polysorbate 80, under the trade name Simulgel® 600, have also proved to be particularly effective according to the invention.

Likewise preferred anionic homopolymers are uncrosslinked and crosslinked polyacrylic acids. Here the preferred crosslinking agents can be allyl ethers of pentaerythritol, sucrose and propylene. Such compounds are commercially available under the trade name Carbopol®, for example.

Further preferred employable anionic polymers are chosen from:
copolymers of vinyl acetate and crotonic acid (as are marketed for example as the commercial product Aristoflex® A 60 with the INCI name VA/Crotonates Copolymer by CIBA in a 60 wt. % conc. dispersion in isopropanol-water),
copolymers of ethyl acrylate and methacrylic acid (as are marketed for example under the trade name Luviflex® Soft with an acid number of 84 to 105 under the INCI name Acrylates Copolymer in a ca. 20 to 30 wt. % conc. dispersion in water by BASF SE),
polyurethanes containing at least one carboxylic group (such as for example a copolymer of isophthalic acid, adipic acid, 1,6-hexane diol, neopentyl glycol and isophorone diisocyanate as is marketed under the trade name Luviset® PUR with the INCI name Polyurethane-1 by BASF SE).

When particularly strong acting thickening anionic polymers are used, care should be taken that the previously cited preferred viscosity criterion of the agent according to the invention is adhered to.

Copolymers of maleic anhydride and methyl vinyl ether, especially those with crosslinks are also color-conserving polymers. A maleic acid-methyl vinyl ether copolymer crosslinked with 1,9-decadiene is commercially available under the trade name Stabileze® QM.

In order to intensify the effect according to the invention, the agents preferably additionally contain at least one surfactant, wherein non-ionic, anionic, cationic, ampholytic surfactants are suitable. The group of ampholytic or amphoteric surfactants includes zwitterionic surfactants and ampholytes. According to the invention, the surfactants can already have an emulsifying action.

Agents according to the invention preferably contain additional surfactants in an amount of 0.01 wt. % to 5 wt. %, particularly preferably 0.05 wt. % to 0.5 wt. %, based on total weight of the agent.

It has proved particularly preferable when the agents according to the invention comprise at least one non-ionic surfactant.

Non-ionic surfactants include a polyol group, a polyalkylene glycol ether group or a combination of polyol ether groups and polyglycol ether groups as the hydrophilic group.

Exemplary compounds of this type are
addition products of 2 to 100 moles ethylene oxide and/or 1 to 5 moles propylene oxide to linear and branched fatty alcohols containing 8 to 30 carbon atoms, to fatty acids containing 8 to 30 carbon atoms and to alkyl phenols containing 8 to 15 carbon atoms in the alkyl group,
methyl or C₂-C₆ alkyl group end blocked addition products of 2 to 50 moles ethylene oxide and/or 1 to 5 moles propylene oxide to linear and branched fatty alcohols with 8 to 30 carbon atoms, to fatty acids with 8 to 30 carbon atoms and to alkyl phenols with 8 to 15 carbon atoms in the alkyl group, such as the commercially available types Dehydrol® LS, Dehydrol® LT (Cognis),
C₁₂-C₃₀ fatty acid mono- and diesters of addition products of 1 to 30 moles ethylene oxide to glycerin,
addition products of 5 to 60 moles ethylene oxide on castor oil and hydrogenated castor oil,
polyol esters of fatty acids, such as the commercial product Hydagen® HSP (Cognis) or Sovermol types (Cognis),
alkoxylated triglycerides,
alkoxylated fatty acid alkyl esters of the formula (E4-I)

R¹CO—(OCH₂CHR²)_{w}OR³ (E4-I)

wherein R¹CO is a linear or branched, saturated and/or unsaturated acyl group containing 6 to 22 carbon atoms, R² is hydrogen or methyl, R³ is linear or branched alkyl groups containing 1 to 4 carbon atoms, and w is a number from 1 to 20,
amine oxides,
mixed hydroxy ethers, such as those described in DE-OS 1 973 8866,
sorbitol esters of fatty acids and addition products of ethylene oxide to sorbitol esters of fatty acids such as polysorbates,
sugar esters of fatty acids and addition products of ethylene oxide to sugar esters of fatty acids,
addition products of ethylene oxide to fatty acid alkanolamides and fatty amines,
sugar surfactants of the alkyl and alkenyl oligoglycosides type according to Formula (E4-II)

R⁴O[G]ₚ (E4-II)
wherein R⁴ is an alkyl or alkenyl group containing 4 to 22 carbon atoms, G is a sugar group containing 5 or 6 carbon atoms, and p is a number from 1 to 10.

Alkylene oxide addition products to saturated, linear fatty alcohols and fatty acids, each with 2 to 100 moles ethylene oxide per mole fatty alcohol or fatty acid, have proved to be quite particularly preferred non-ionic surfactants. Similarly, preparations with excellent properties are obtained when they contain C₁₂-C₃₀ fatty acid mono- and diesters of addition products of 1 to 30 moles ethylene oxide to glycerin and/or addition products of 5 to 60 moles ethylene oxide to castor oil and hydrogenated castor oil as the non-ionic surfactants.

Suitable anionic surfactants include all anionic surface-active materials suitable for use on the human body. They are characterized by a water solubilizing anionic group, such as a carboxylate, sulfate, sulfonate or phosphate group and a lipophilic alkyl group containing about 8 to 30 carbon atoms. In addition, the molecule may contain glycol or polyglycol ether groups, ester, ether and amide groups as well as hydroxyl groups. Exemplary suitable anionic surfactants are, each in the form of the sodium, potassium and ammonium as well as the mono, di and trialkanolammonium salts containing 2 to 4 carbon atoms in the alkanol group,
linear and branched fatty acids with 8 to 30 carbon atoms (soaps),
ether carboxylic acids of the formula R—O—(CH₂—CH₂O)ₓ—CH₂—COOH, wherein R is a linear alkyl group with 8 to 30 carbon atoms and x=0 or 1 to 16,
acyl sarcosides with 8 to 24 carbon atoms in the acyl group,
acyl taurides with 8 to 24 carbon atoms in the acyl group,
acyl isethionates with 8 to 24 carbon atoms in the acyl group,
mono- and dialkyl esters of sulfosuccinic acid with 8 to 24 carbon atoms in the alkyl group and mono-alkyl polyoxyethyl esters of sulfosuccinic acid with 8 to 24 carbon atoms in the alkyl group and 1 to 6 oxyethylene groups,
linear alkane sulfonates containing 8 to 24 carbon atoms,
linear alpha-olefin sulfonates containing 8 to 24 carbon atoms,
alpha-sulfo fatty acid methyl esters of fatty acids containing 8 to 30 carbon atoms,
alkyl sulfates and alkyl polyglycol ether sulfates of the Formula R—O(CH₂—CH₂O)ₓ—OSO₃H, wherein R is preferably a linear alkyl group containing 8 to 30 carbon atoms and x=0 or 1 to 12,
mixtures of surface active hydroxyl sulfonates, sulfated hydroxyalkyl polyethylene glycol ethers and/or hydroxyalkylene propylene glycol ethers,
sulfonates of unsaturated fatty acids with 8 to 24 carbon atoms and 1 to 6 double bonds,
esters of tartaric acid and citric acid with alcohols, which represent the addition products of about 2-15 molecules of ethylene oxide and/or propylene oxide on fatty alcohols containing 8 to 22 carbon atoms,
sulfated fatty acid alkylene glycol esters of Formula (E1-II)

R⁷CO(AIkO)ₙSO₃M (E1-II)
wherein R⁷CO— is a linear or branched, aliphatic, saturated and/or unsaturated acyl group containing 6 to 22 carbon atoms, Alk is CH₂CH₂, CHCH₃CH₂ and/or CH₂CHCH₃, n is a number from 0.5 to 5 and M is a cation, as are described in DE-OS 197 36 906,
amido ether carboxylic acids,
condensation products of C₈-C₃₀ fatty alcohols with protein hydrolyzates and/or amino acids and their derivatives, known to one skilled in the art as albumin fatty acid condensates, such as the Lamepon® types, Gluadin® types, Hostapon® KCG or the Amisoft® types.

Preferred anionic surfactants are alkyl sulfates and ether carboxylic acids with 10 to 18 carbon atoms in the alkyl group and up to 12 glycol ether groups in the molecule, sulfosuccinic acid mono and dialkyl esters with 8 to 18 C atoms in the alkyl group and sulfosuccinic acid mono-alkyl polyoxyethyl esters with 8 to 18 C atoms in the alkyl group and 1 to 6 oxyethylene groups, monoglycerin disulfates, alkyl- and alkenyl ether phosphates as well as albumin fatty acid condensates.

Zwitterionic surfactants are those surface-active compounds having at least one quaternary ammonium group and at least one —COO⁽⁻⁾ or —SO₃⁽⁻⁾ group in the molecule. Particularly suitable zwitterionic surfactants are betaines such as N-alkyl-N,N-dimethylammonium glycinates, for example, cocoalkyl-dimethylammonium glycinate, N-acyl-aminopropyl-N,N-dimethylammonium glycinate, for example, coco-acylaminopropyl-dimethylammonium glycinate, and 2-alkyl-3-carboxymethyl-3-hydroxyethyl-imidazolines each with 8 to 18 carbon atoms in the alkyl or acyl group, as well as cocoacyl-aminoethylhydroxyethylcarboxymethyl glycinate. A preferred zwitterionic surfactant is the fatty acid amide derivative known under the INCI name Cocamidopropyl Betaine.

Ampholytes include surface-active compounds that, apart from a C₈₋₂₄ alkyl or acyl group, have at least one free amino group and at least one —COON or —SO₃H group in the molecule and are able to form internal salts. Examples of suitable ampholytes are N-alkylglycines, N-alkyl propionic acids, N-alkylamino butyric acids, N-alkylimino dipropionic acids, N-hydroxyethyl-N-alkylamidopropylglycines, N-alkyltaurines, N-alkylsarcosines, 2-alkylamino propionic acids and alkylamino acetic acids, each with about 8 to 24 carbon atoms in the alkyl group. Particularly preferred ampholytes are N-cocoalkylamino propionate, the cocoacylaminoethylamino propionate and the C₁₂₋₁₈ acyl sarcosine.

Agents according to the invention include ingredients or active substances in a cosmetically acceptable carrier. Preferred cosmetically acceptable carriers are aqueous, alcoholic or aqueous alcoholic media containing preferably at least 10 wt. % water, based on total composition. In particular, lower alcohols containing 1 to 4 carbon atoms, such as ethanol and isopropanol, which are typically used for cosmetic purposes, can be comprised as alcohols.

Organic solvents or mixture of solvents having a boiling point of less than 400° C. can be used as additional co-solvents in an amount of 0.1 to 15 weight percent, preferably 1 to 10 weight percent, based on total agent. Particularly suitable additional co-solvents are unbranched or branched hydrocarbons such as pentane, hexane, isopentane and cyclic hydrocarbons such as cyclopentane and cyclohexane. Additional, preferred water-soluble solvents are glycerin, ethylene glycol and propylene glycol in an amount of up to 30 wt. % based on total agent.

In particular, the addition of glycerin and/or propylene glycol and/or polyethylene glycol and/or polypropylene glycol increases the flexibility of the polymer film that is formed when the agent according to the invention is used. Consequently, if a more flexible hold is desired, then the agents according to the invention preferably comprise 0.01 to 30 wt. % glycerin and/or propylene glycol and/or polyethylene glycol and/or polypropylene glycol, based on the total agent.

The agents preferably exhibit a pH of 2 to 11. The pH range is particularly preferably from 2 to 8. In the context of this publication, pH data refers to the pH at 25° C. unless otherwise stated.

Agents according to the invention can also include auxiliaries and additives usually incorporated into conventional styling agents. In particular, care products may be mentioned as suitable auxiliaries and additives. According to the invention, at least one silicone oil and/or at least one silicone gum is preferably employed as a care substance.

Suitable silicone oils or gums according to the invention are dialkyl and alkylarylsiloxanes such as dimethylpolysiloxane and methylphenylpolysiloxane, as well as their alkoxylated, quaternized or also anionic derivatives. Cyclic and linear polydialkylsiloxanes, their alkoxylated and/or aminated derivatives, dihydroxypolydimethylsiloxanes and polyphenylalkylsiloxanes are preferred.

Silicone oils afford the most varied effects. Thus, for example, they simultaneously influence dry and wet combability, the feel of dry and wet hair, as well as the gloss. The term “silicone oils” is understood by one skilled in the art to mean organosilicon compounds with a plurality of structures. In the first instance they include Dimethiconols.

The following commercial products are examples of such products: Botanisil NU-150M (Botanigenics), Dow Corning 1-1254 Fluid, Dow Corning 2-9023 Fluid, Dow Corning 2-9026 Fluid, Ultrapure Dimethiconol (Ultra Chemical), Unisil SF-R (Universal Preserve), X-21-5619 (Shin-Etsu Chemical Co.), Abil OSW 5 (Degussa Care Specialties), ACC DL-9430 Emulsion (Taylor Chemical Company), AEC Dimethiconol & Sodium Dodecylbenzene sulfonate (A & E Connock (Perfumery & Cosmetics) Ltd.), B C Dimethiconol Emulsion 95 (Basildon Chemical Company, Ltd.), Cosmetic Fluid 1401, Cosmetic Fluid 1403, Cosmetic Fluid 1501, Cosmetic Fluid 1401 DC (all from Chemsil Silicones, Inc.), Dow Corning 1401 Fluid, Dow Corning 1403 Fluid, Dow Corning 1501 Fluid, Dow Corning 1784 HVF Emulsion, Dow Corning 9546 Silicone Elastomer Blend (all from Dow Corning Corporation), Dub Gel S1 1400 (Stearinerie Dubois Fils), HVM 4852 Emulsion (Crompton Corporation), Jeesilc 6056 (Jeen International Corporation), Lubrasil, Lubrasil DS (both from Guardian Laboratories), Nonychosine E, Nonychosine V (both from Exsymol), SanSurf Petrolatum-25, Satin Finish (both from Collaborative Laboratories, Inc.), Silatex-D30 (Cosmetic Ingredient Resources), Silsoft 148, Silsoft E-50, Silsoft E-623 (all from Crompton Corporation), SM555, SM2725, SM2765, SM2785 (all from GE Silicones), Taylor T-SiI CD-1, Taylor TME-4050E (all from Taylor Chemical Company), TH V 148 (Crompton Corporation), Tixogel CYD-1429 (Sud-Chemie Performance Additives), Wacker-Belsil CM 1000, Wacker-Belsil CM 3092, Wacker-Belsil CM 5040, Wacker-Belsil DM 3096, Wacker-Belsil DM 3112 VP, Wacker-Belsil DM 8005 VP, Wacker-Belsil DM 60081 VP (all from Wacker-Chemie GmbH).

Dimethicones form the second group of silicones that can be used according to the invention. They can be linear, branched, cyclic, or cyclic and branched. Dimethicone copolyols form a further group of suitable silicones. Suitable Dimethicone copolyols are commercially available and marketed, for example, by Dow Corning under the trade name Dow Corning® 5330 Fluid.

Naturally, the Dimethiconols, Dimethicones and/or Dimethicone copolymers can be present as an emulsion. When the Dimethiconols, Dimethicones and/or Dimethicone copolyols are used as an emulsion, then according to the invention, the droplet size of the emulsified particles ranges from 0.01 to 10 000 μm, preferably 0.01 to 100 particularly preferably 0.01 to 20 μm and quite particularly preferably 0.01 to 10 μm. Particle size is determined here according to the light scattering method.

Further suitable silicones are amino-functional silicones, especially the silicones compiled under the INCI name Amodimethicone. Consequently, it is inventively preferred when the agents additionally contain at least one amino-functional silicone. These refer to silicones having at least one, optionally substituted, amino group. These silicones are designated as Amodimethicones according to INCI nomenclature and are available, for example, in the form of an emulsion as the commercial product Dow Corning® 939 or as the commercial product Dow Corning® 949 in mixture with a cationic and a non-ionic surfactant.

Preferably, those amino functional silicones are employed that have an amine number of 0.25 meq/g or greater, preferably of 0.3 meq/g or greater, and particularly preferably of 0.4 meq/g or greater. The amine number stands for the milli-equivalents of amine per gram of the amino functional silicone. It can be measured by titration and can also be reported with the unit mg KOH/g.

The agents preferably contain silicones in amounts of 0.01 wt. % to 15 wt. %, preferably 0.05 to 2 wt. %, based on total weight of the agent.

The composition can optionally comprise at least one protein hydrolyzate and/or one of its derivatives as a care substance of another compound class. Agents according to the invention contain protein hydrolyzates, for example, in concentrations of 0.01 wt. % to 20 wt. %, preferably 0.05 wt. % up to 15 wt. % and quite particularly preferably in amounts of 0.05 wt. % up to 5 wt. %, based on total weight of the end-use preparation.

The agents can further contain at least one vitamin, one provitamin, one vitamin precursor and/or one of their derivatives as the care substance.

According to the invention, those vitamins, provitamins and vitamin precursors which are normally classified in the groups A, B, C, E, F and H are preferred. Retinol (vitamin A₁) as well as 3,4-didehydroretinol, (vitamin A₂) belong in the group of substances designated as vitamin A. The vitamin B group or the vitamin B complex include inter alia vitamin B₁ (thiamine), vitamin B₂ (riboflavin), vitamin B₃ (nicotinic acid and/or nicotinic acid amide (niacinamide)), vitamin B₅ (pantothenic acid, panthenol and pantolactone), vitamin B₆ (pyridoxine as well as pyridoxamine and pyridoxal). Other representatives of the abovementioned vitamins are vitamin C (ascorbic acid), vitamin E (tocopherols, especially α-tocopherol), vitamin F (linoleic acid and/or linolenic acid), vitamin H.

Agents according to the invention preferably contain vitamins, provitamins and vitamin precursors from groups A, B, C, E and H. Panthenol, pantolactone, pyridoxine and its derivatives as well as nicotinamide and biotin are especially preferred. D-panthenol is quite particularly preferably employed as a care substance, optionally in combination with at least one of the abovementioned silicone derivatives.

Thus, if a particularly flexible hold is desired, then the agents can contain panthenol instead of or in addition to glycerin and/or propylene glycol. In a preferred embodiment, the agents contain panthenol, preferably in an amount of 0.05 to 10 wt. %, particularly preferably 0.1 to 5 wt. %, based on total weight of the agent.

The agents can further contain at least one plant extract as a care substance. Usually, these extracts are manufactured by extraction of the whole plant. In individual cases, however, it can also be preferred to produce the extracts solely from blossoms and/or leaves of the plant. According to the invention, extracts from green tea, oak bark, stinging nettle, hamamelis, hops, henna, chamomile, burdock root, field horsetail, hawthorn, linden flowers, almonds, aloe vera, spruce needles, horse chestnut, sandal wood, juniper, coconut, mango, apricot, lime, wheat, kiwi, melon, orange, grapefruit, sage, rosemary, birch, malva, lady's smock, common yarrow, thyme, lemon balm, rest-harrow, coltsfoot, marshmallow (althea), meristem, ginseng and ginger are preferred.

The agent can further contain at least one lipid as a care substance. According to the invention, suitable lipids are phospholipids, for example, soy lecithin, egg lecithin and cephalins as well as the substances known under the INCI names Linoleamidopropyl PG-Dimonium Chloride Phosphate, Cocamidopropyl PG-Dimonium Chloride Phosphate and Stearamidopropyl PG-Dimonium Chloride Phosphate. These are commercialized, for example, by the Mona Company under the trade names Phospholipid EFA®, Phospholipid PTC® and Phospholipid SV®. The agents preferably contain lipids in amounts of 0.01 to 10 wt. %, particularly 0.1 to 5 wt. %, based on total weight of the end-use preparation.

Oil bodies are also suitable as a care substance.

Natural and synthetic cosmetic oil bodies include:
vegetal oils. Examples of such oils are sunflower oil, olive oil, soya oil, rapeseed oil, almond oil, jojoba oil, orange oil, wheat germ oil, peach stone oil and the liquid parts of coconut oil. Other triglyceride oils such as liquid fractions of beef tallow as well as synthetic triglyceride oils are also suitable.
liquid paraffin oils, isoparaffin oils and synthetic hydrocarbons as well as di-n-alkyl ethers containing a total of 12 to 36 carbon atoms, particularly 12 to 24 carbon atoms, such as di-n-octyl ether, di-n-decyl ether, di-n-nonyl ether, di-n-undecyl ether, di-n-dodecyl ether, n-hexyl n-octyl ether, n-octyl n-decyl ether, n-decyl n-undecyl ether, n-undecyl n-dodecyl ether and n-hexyl n-undecyl ether and di-tert.butyl ether, diisopentyl ether, di-3-ethyldecyl ether, tert.butyl n-octyl ether, isopentyl n-octyl ether and 2-methylpentyl n-octyl ether. The commercial products of the compounds 1,3-di-(2-ethylhexyl)cyclohexane (Cetiol® S) and di-n-octyl ether (Cetiol® OE) can be preferred.
Ester oils. Ester oils refer to the esters of C₆-C₃₀ fatty acids with C₂-C₃₀ fatty alcohols. The monoesters of fatty acids with alcohols containing 2 to 24 carbon atoms are preferred. According to the invention, isopropyl myristate (Rilanit® IPM), isononanoic acid C16-18 alkyl ester (Cetiol® SN), 2-ethylhexyl palmitate (Cegesoft® 24), stearic acid 2-ethylhexyl ester (Cetiol® 868), cetyl oleate, glycerine tricaprylate, cocofatty alcohol caprinate/-caprylate (Cetiol® LC), n-butyl stearate, oleyl erucate (Cetiol® J 600), isopropyl palmitate (Rilanit® IPP), oleyl oleate (Cetiol®), lauric acid hexyl ester (Cetiol® A), di-n-butyl adipate (Cetiol® B), myristyl myristate (Cetiol® MM), cetearyl isononanoate (Cetiol® SN), oleic acid decyl ester (Cetiol® V) are particularly preferred.
Dicarboxylic acid esters such as di-n-butyl adipate, di-(2-ethylhexyl) adipate, di-(2-ethylhexyl) succinate and di-isotridecyl acetate as well as diol esters such as ethylene glycol dioleate, ethylene glycol di-isotridecanoate, propylene glycol di(2-ethylhexanoate), propylene glycol di-isostearate, propylene glycol di-pelargonate, butane diol di-isostearate, neopentyl glycol dicaprylate,
symmetrical, unsymmetrical or cyclic esters of carbon dioxide with fatty alcohols, for example, as described in DE-OS 197 56 454, glycerine carbonate or dicaprylyl carbonate (Cetiol® CC),
trifatty acid esters of saturated and/or unsaturated linear and/or branched fatty acids with glycerin,
fatty acid partial glycerides, under which are understood monoglycerides, diglycerides and their industrial mixtures. When using industrial products, minor amounts of triglycerides may still be contained as a result of the production process. The partial glycerides preferably correspond to the Formula (D4-1),

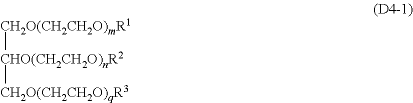

where R¹, R² and R³ are, independently of each other, hydrogen or a linear or branched, saturated and/or unsaturated acyl group containing 6 to 22 carbon atoms, preferably 12 to 18 carbon atoms, with the proviso that at least one of these groups is an acyl group and at least one of these groups is hydrogen. The sum of (m+n+q) is 0 or a number from 1 to 100, preferably 0 or 5 to 25. Preferably, R¹ is an acyl group, R² and R³ are hydrogen, and the sum of (m+n+q) is 0. Typical examples are mono- and/or diglycerides based on caproic acid, caprylic acid, 2-ethylhexanoic acid, capric acid, lauric acid, isotridecanoic acid, myristic acid, palmitic acid, palmitoleic acid, stearic acid, isostearic acid, oleic acid, elaidic acid, petroselic acid, linoleic acid, linolenic acid, elaeostearic acid, arachidonic acid, gadoleic acid, behenic acid and erucic acid as well as their industrial mixtures. Oleic acid monoglycerides are preferably employed.

The amount of natural and synthetic cosmetic oil bodies in the agents is usually 0.1 to 30 wt. %, based on total weight of the end-use preparation, preferably 0.1 to 20 wt. % and particularly 0.1 to 15 wt. %.

By addition of a UV filter, both the agent itself as well as the treated fibers can be protected against damage from UV radiation. Consequently, at least one UV filter is preferably added to the agent. Suitable UV filters are generally not limited with respect to their structure and their physical properties. Indeed, all UV filters that can be used in the cosmetic field and having an absorption maximum in the UVA (315-400 nm), UVB (280-315 nm) or UVC (<280 nm) regions are suitable. UV filters having an absorption maximum in the UVB region, especially in the range from about 280 to about 300 nm, are particularly preferred. Preferred UV-filters are chosen from substituted benzophenones, p-aminobenzoates, diphenylacrylates, cinnamates, salicylates, benzimidazoles and o-aminobenzoates.

The agent usually contains UV filters in amounts of 0.01 to 5 wt. %, based on total weight of the end-use preparation. Quantities of 0.1-2.5 wt. % are preferred.

In a particular embodiment, the agent further contains one or more substantive dyes. Application of the agent then allows the treated keratinic fiber not only to be temporarily styled, but also be dyed at the same time. This can be particularly desirable when only a temporary dyeing is desired, for example, with flamboyant fashion colors that can be subsequently removed from the keratinic fibers by simply washing them out. Substantive dyes typically used are nitrophenylenediamines, nitroamino phenols, azo dyes, anthraquinones, indophenols or cationic substantive dyes. Particularly preferred cationic dyes are
cationic triphenylmethane dyes such as Basic Blue 7, Basic Blue 26, Basic Violet 2 and Basic Violet 14,
aromatic systems substituted by a quaternary nitrogen group, such as Basic Yellow 57, Basic Red 76, Basic Blue 99, Basic Brown 16 and Basic Brown 17, and
substantive dyes having a heterocycle possessing at least one quaternary nitrogen atom. Dyes also known under the names Basic Yellow 87, Basic Orange 31 and Basic Red 51 are quite particularly preferred cationic substantive dyes.

Cationic substantive dyes commercialized under the trade name Arianor® are likewise quite particularly preferred cationic substantive dyes according to the invention. In addition, compositions according to the invention can also contain naturally occurring dyestuffs present, for example, in henna red, henna neutral, henna black, chamomile leaves, sandalwood, black tea, alder buckthorn bark, sage, logwood, madder root, cachou, cedar and alkanet root.

Inventive agents according to this embodiment contain substantive dyes preferably in an amount of 0.001 to 20 wt. %, based on total weight of the agent.

Preferably, agents according to the invention are exempt from oxidation dye precursors. Oxidation dye precursors are divided into developer components and coupler components. Under the influence of oxidizing agents or from atmospheric oxygen, the developer components form the actual dyes among each other or by coupling with one or more coupler components.

Formulation of the inventive agents can be in all forms typical for styling agents, for example, in the form of solutions that can be applied as hair water or pump or aerosol spray onto the hair, in the form of creams, emulsions, waxes, gels or surfactant-containing foaming solutions or other preparations, which are suitable for application on the hair. The agents are preferably packaged as a hair cream or hair gel, especially as a hair gel.

A second subject matter of the invention is the use of the agent according to the invention for the temporary shaping of hair and/or for hair care.

Agents according to the invention and products containing these agents, especially hair gels or hair creams, provide the treated hair with a very strong, long-lasting hold to the hairstyle while keeping the hair flexible. If the agent is in the form of a hair gel, then the gel has a pasty consistency that can be uniformly dispersed on the hair without any dripping.

It is inventively preferred to use the agent of the first subject matter of the invention as a leave-on hair treatment agent.

A third subject matter of the invention is a method for treating keratin-containing fibers, especially human hair, wherein an agent of the first subject matter is applied onto the keratin-containing fibers.

It is inventively preferred when the keratin-containing fibers are styled before, during or after the application of the agent according to the invention.

Furthermore, it is inventively preferred not to rinse out the agent according to the invention from the keratin-containing fibers.

The following examples are intended to illustrate the subject matter of the present invention in more detail, without limiting it in any way.

### EXAMPLES—

Unless otherwise stated, all quantities are in weight percent. Inventive styling gels A to D according to the following Table were manufactured—

| Raw Materials | A | B | C | D |
|---|---|---|---|---|
| Benzophenone-4 | 0.05 | 0.05 | 0.05 | 0.05 |
| Synthalen K 1 | 0.40 | — | 0.40 | 0.40 |
| Carbopol Ultrez 21 2 | — | 0.30 | — | — |
| Neolone TE 4 | 0.50 | 0.50 | 0.50 | 0.50 |
| D-Panthenol | 0.15 | 0.15 | 0.15 | 0.15 |
| Aquastyle ® 300 5 | 10.00 | 10.00 | 10.00 | 10.00 |
| Sorbitol | 3.00 | 3.00 | 3.00 | 3.00 |
| Polyvinyl pyrrolidone | — | — | 1.00 | — |
| Luviskol VA 64 W 6 | — | — | — | 2.00 |
| PEG-40 hydrogenated castor oil | 0.55 | 0.55 | 0.55 | 0.55 |
| Perfume | 0.10 | 0.10 | 0.10 | 0.10 |
| Water | ad 100 | ad 100 | ad 100 | ad 100 |

1 Polyacrylic acid (ca. 89% active substance content; INCI name: Carbomer) (3V Sigma)
2 Crosslinked acrylic acid copolymer, white powder (INCI name: Acrylates/C10-30 Alkylacrylate Crosspolymer) (Noveon),
³Solution of ca. 2 wt. % 2-methyl-4-isothiazolin-3-one in ca. 83.5 wt. % phenoxyethanol, 12.5 wt. % propane-1,2-diol and ca. 2 wt. % water (INCI name: Phenoxyethanol, Methylisothiazolinone) (Rohm & Haas),
4 N,N,N′,N′,-Tetrakis-(2-hydroxypropyl)ethylenediamine (INCI name: Tetrahydroxypropyl Ethylenediamine) (BASF)
5 Copolymer of N-vinyl pyrrolidone/N-vinyl caprolactam/N-(3-dimethylaminopropyl)methacrylamide and 3-(methacryloylamino)propyl-lauryl-dimethylammonium chloride (active substance 30 wt. % in water/ethanol, INCI name: Polyquaternium-69) (ISP),
6 Copolymer of N-vinyl pyrrolidone and vinyl acetate (60:40) (48-52% active substance in water, INCI name: VP/VA Copolymer) (BASF)

Each Formulation A to D was tested on the hair of a test person. An adequate amount of gel was placed onto the palm of the hand and dispersed in the hair. The hair was then dressed and styled. The resulting hairstyle had a flexible hold without the formation of film plaques. Formulations C and D imparted an ultra-strong hold. Formulation D was slightly turbid/discolored and imparted a worse hold to the hairstyle than Formulation C.

We claim:

1. Agent for treating keratin-containing fibers comprising, in a cosmetically acceptable carrier:
(a) at least one amphiphilic, cationic polymer having at least one structural unit of Formula (I), at least one structural unit of Formula (II), at least one structural unit of Formula (III), and at least one structural unit of Formula (IV)— 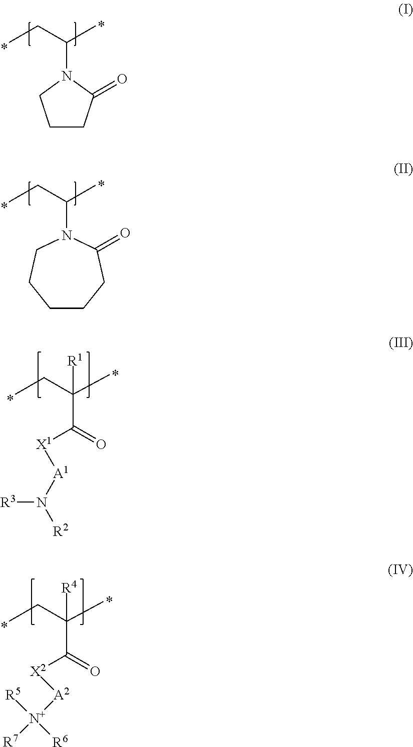
wherein
R¹ and R⁴ are, independently of one another, a hydrogen atom or a methyl group,
X¹ and X² are, independently of one another, an oxygen atom or an NH group,
A¹ and A² are, independently of one another, an ethane-1,2-diyl, propane-1,3-diyl or butane-1,4-diyl group,
R², R³, R⁵ and R⁶ are, independently of one another, a (C₁ to C₄) alkyl group,
R⁷ is a (C₈ to C₃₀) alkyl group,
(b) at least one polyol, and
(c) as an additional film-forming and/or setting polymer a non-ionic polymer, selected from homopolymers of N-vinyl pyrrolidone and/or from copolymers of N-vinyl pyrrolidone.

2. Agent according to claim 1 wherein A¹ and A² are, independently of one another, ethane-1,2-diyl or propane-1,3-diyl.

3. Agent according to claim 1 wherein R², R³, R⁵ and R⁶ are, independently of one another, methyl or ethyl.

4. Agent according to claim 1 wherein R⁷ is decyl, undecyl, dodecyl, tridecyl, tetradecyl, hexadecyl, octadecyl, eicosyl or docosyl.

5. Agent according to claim 1 wherein the at least one amphiphilic, cationic polymer is chosen from at least one polymer having a structural unit according to the following Formulae (I), (II), (III-8) or (IV-8) 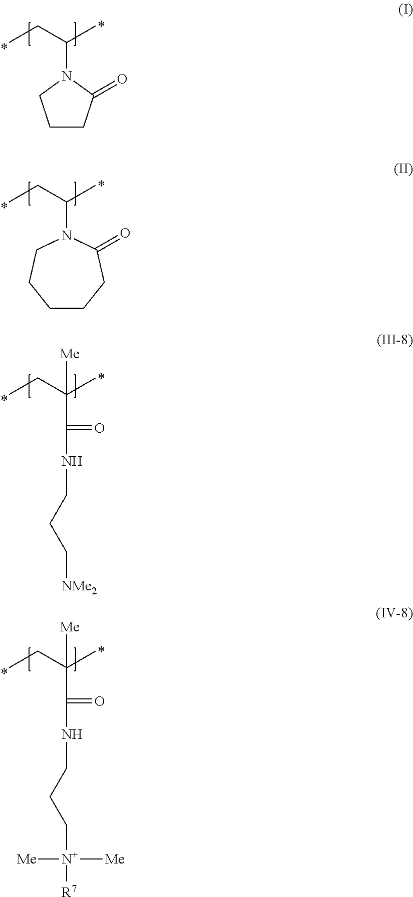
wherein R⁷ is a (C₈ to C₃₀) alkyl group.

6. Agent according to claim 1 wherein the at least one amphiphilic, cationic polymer is present in an amount of 0.05 wt. % to 15.0 wt. %, based on total weight of the agent.

7. Agent according to claim 1 wherein the at least one polyol is present in an amount of 0.05 wt. % to 15.0 wt. %, based on total weight of the agent.

8. Agent according to claim 1 wherein the at least one amphiphilic, cationic polymer (a) and the at least one polyol (b) are present in a molar ratio of 1 to 50 to 50 to 1.

9. Agent according to claim 1 wherein the polyols have a chemical structure consisting of carbon atoms, hydrogen atoms and oxygen atoms and has a hydrocarbon backbone, onto which at least two hydroxyl groups bond and onto which the oxygen atoms bond as hydroxyl groups and/or as ether functionalities onto the hydrocarbon backbone.

10. Agent according to claim 1 wherein the polyols are solid at 25° C. and a pressure of 1 atm.

11. Agent according to claim 1 wherein the at least one polyol (b) is chosen from sorbitol, inositol, xylitol, mannitol, gluconolactone, glucuronic acid, 1,2,6-hexane triol, hydroxyethylsorbitol, phytantriol, hydroxypropylphytantriol, lactitol, maltitol, pentaerythritol, polyglycerin-3, glucose, lactose, maltose, polyglycerin-4, polyglycerin-6, polyglycerin-10, polyglycerin-20, polyglycerin-40, tripropylene glycol, especially sorbitol.

12. Agent according to claim 1 wherein the film-forming and/or setting polymers (c) is present in an amount of 0.01 wt. % to 20 wt. %, based on total weight of the agent.
